(19) United States
(12) Patent Application Publication
(10) Pub. No.: US 2008/0255373 A1
(43) Pub. Date: Oct. 16, 2008

(54) PROCESS FOR THE PRODUCTION OF GLYCEROL CARBONATE ESTERS

(76) Inventor: Alfred Westfechtel, Hilden (DE)

Correspondence Address: SMITH MOORE LEATHERWOOD LLP, P.O. BOX 21927, GREENSBORO, NC, 27420, US

(21) Appl. No.: 11/764,982

(22) Filed: Jun. 19, 2007

(30) Foreign Application Priority Data
Jun. 22, 2006 (EP) 06012792

(51) Int. Cl. C07D 317/34

(52) U.S. Cl. 549/229

(57) ABSTRACT

The invention relates to a process for the production of glycerol carbonate esters corresponding to formula (I):

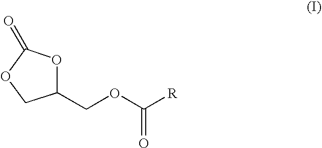

in which R is hydrogen or an alkyl or alkenyl group containing 1 to 23 carbon atoms, which may be linear or branched, characterized in that a compound corresponding to formula (II):

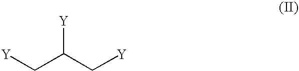

in which Y is a group —O—CO—Z and Z is hydrogen or an alkyl or alkenyl group containing 1 to 3 carbon atoms, which may be linear or branched, is transesterified with dimethyl carbonate or diethyl carbonate and an alkyl ester corresponding to formula (IV):

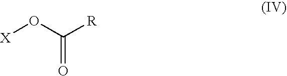

in which R is as defined above and X is an alkyl or alkenyl group containing 1 to 8 carbon atoms, which may be linear or branched, with the proviso that the group R of compound (IV) is different from the group Z of compound (II), in the presence of a transesterification catalyst.

### RELATED APPLICATIONS

This application claims priority from European Patent Application EP0601279 2.5 filed Jun. 22, 2006, the entire contents of which are incorporated herein by reference.

### FIELD OF THE INVENTION

This invention relates to a process for the production of glycerol carbonate esters.

### BACKGROUND OF THE INVENTION

Esters of glycerol carbonate have been relatively little reported. According to Zephirin Mouloungui et al., Eur. J. Lipid Sci. Technol., 103 (2001) 216-222, the long-chain carbonate esters are obtained by reaction of glycerol carbonate with acid chlorides. In a review of cyclic carbonate-functional polymers in Progress in Organic Coatings, 47 (2003), 77-86, Dean C. Webster presents several synthesis routes for glycerol carbonate methacrylate, including the transesterification of glycerol carbonate with methyl methacrylate. U.S. Pat. No. 3,225,063 describes the production of carbonate esters with cyclic anhydrides in the form of acidic semiesters.

Special publications are devoted to acrylates and methacrylates as unsaturated esters of glycerol carbonate. Thus, U.S. Pat. No. 2,979,514 describes the reaction of acid chlorides and glycerol carbonate. Large excesses of reagents and solvents or entraining agents are used for the reaction. DE 10355830 A1 describes a process in which metal chelate catalysts are used for the transesterification of glycerol carbonate with methyl methacrylate. Here, too, a large excess of acrylate is used.

WO 93109111 A2 discloses a process for the production of glycerol carbonate esters in which glycerol esters with C₁₋₂₃ fatty acids, more particularly triglycerides based on such fatty acids, are reacted with a carbonate, more particularly dimethyl carbonate or diethyl carbonate, in the presence of a catalyst.

### BRIEF DESCRIPTION OF THE INVENTION

The problem addressed by the present invention was to provide a new process for the production of glycerol carbonate esters.

The present invention relates to a process for the production of glycerol carbonate esters corresponding to formula (I):

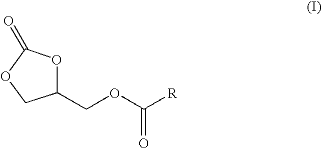

in which R is hydrogen or an alkyl or alkenyl group containing 1 to 23 carbon atoms, which may be linear or branched, wherein, a compound corresponding to formula (II):

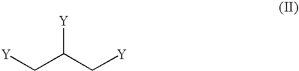

in which Y is a group —O—CO—Z and Z is hydrogen or an alkyl or alkenyl group containing 1 to 3 carbon atoms, which may be linear or branched, is transesterified with dimethyl carbonate or diethyl carbonate and an alkyl ester corresponding to formula (IV):

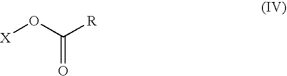

in which R is as defined above and X is an alkyl or alkenyl group containing 1 to 8 carbon atoms, which may be linear or branched, with the proviso that the group R of compound (IV) is different from the group Z of compound (II), in the presence of a transesterification catalyst.

It is expressly pointed out that the word “or” in the expression “dimethyl carbonate or diethyl carbonate” relates only to the two carbonates. In other words, three reaction components are reacted with one another in the process according to the invention, namely

compound (II),

dimethyl carbonate or diethyl carbonate,

compound (IV)

### DETAILED DESCRIPTION OF THE INVENTION

In one embodiment, the process according to the invention is carried out in two steps. In this embodiment, the invention relates to a process for the production of glycerol carbonate esters corresponding to formula (I)

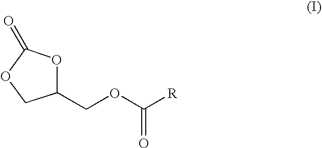

in which R is hydrogen or an alkyl or alkenyl group containing 1 to 23 carbon atoms, which may be linear or branched, characterized in that, in a first step, a compound corresponding to formula (II):

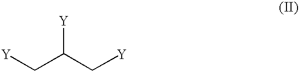

in which Y is a group —O—CO—Z and Z is hydrogen or an alkyl or alkenyl group containing 1 to 3 carbon atoms, which may be linear or branched, is reacted with dimethyl carbonate or diethyl carbonate to form a compound corresponding to formula (III):

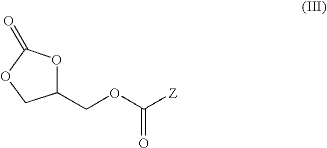

in which Z is as defined above,
and, in a second step, the compound (III) is transesterified with an alkyl ester corresponding to formula (IV):

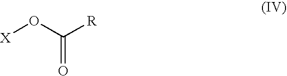

in which R is as defined above and X is an alkyl or alkenyl group containing 1 to 8 carbon atoms, which may be linear or branched, with the proviso that the group R of compound (IV) is different from the group Z of compound (II),
in the presence of a transesterification catalyst.

Although readily understood by the expert, the meaning of the proviso that “the group R of compound (IV) is different from the group Z of compound (II)” is explained in the following. This proviso means that
If the target compound (1) with R=hydrogen is to be produced, compounds (IV) where R is hydrogen have to be used; accordingly, the group Z of the compounds (II) cannot be hydrogen.
if the target compound (I) with R=methyl is to be produced, compounds (IV) where R is methyl have to be used; accordingly, the group Z of the compounds (II) cannot be methyl.
If the target compound (I) with R=ethyl is to be produced, compounds (IV) where R is ethyl have to be used; accordingly, the group Z of the compounds (II) cannot be ethyl.
If the target compound (I) with R=n-propyl is to be produced, compounds (IV) where R is n-propyl have to be used; accordingly, the group Z of the compounds (II) cannot be n-propyl.
If the target compound (I) with R=i-propyl is to be produced, compounds (IV) where R is i-propyl have to be used; accordingly, the group Z of the compounds (II) cannot be i-propyl.
If the target compound (I) with R═—CH═CH₂ is to be produced, compounds (IV) where R is —CH═CH₂ have to be used; accordingly, the group Z of the compounds (II) cannot be —CH═CH₂.
If the target compound (I) with R═—C(CH₃)═CH₂ is to be produced, compounds (IV) where R is —C(CH₃)═CH₂ have to be used; accordingly, the group Z of the compounds (II) cannot be —C(CH₃)═CH₂.

The reason for the proviso becomes clear when the above-mentioned 2-step character of the process according to the invention is considered. In step 1, the intermediate compound (III) is obtained in which the substituent Z is formally exchanged for the substituent R in step 2, the target compound (I) being formed. If, now, Z and R were identical in the actual reaction, no compound different from the intermediate compound (III) could be formed.

In a preferred embodiment of the process according to the invention in both the one-step and the two-step variant, the compounds CH₃—O—CO—Z or CH₃—CH₂—O—CO—Z and X—O—CO—Z formed in the course of the reaction are continuously removed from the reaction mixture, more particularly by distillation. This continuous removal by distillation has the advantage that the reaction can be carried out under moderate conditions and gives light-colored target products (I),

The process according to the invention has several advantages. Inexpensive, readily obtainable starting materials are used. The compounds CH₃—O—CO—Z or CH₃—CH₂—O—C—Z and X—O—CO—Z (secondary products) formed in the course of the reaction on the one hand are easy to remove and, on the other hand, can be used as products of value (methyl acetate for example can be used as a solvent). For the rest, the process according to the invention can be carried out under moderate conditions.

Basically, the choice of the transesterification catalysts is not critical. In principle, any transesterification catalyst may be used. Particularly suitable transesterification catalysts are alcoholates, such as sodium methylate, potassium methylate, or hydroxides, such as sodium hydroxide and potassium hydroxide, and alkali metal carbonates. The catalysts are preferably used in a quantity of 0.01 to 5% and more particularly 0.5 to 1%, based on the reaction mixture as a whole. In the two-step variant, the same catalyst is preferably used in both steps.

Basically, the reaction temperature is not critical. The reaction is preferably carried out between 40 and 150° C. The temperature range from 60 to 110° C. is particularly preferred.

The reactants are preferably reacted with one another in stoichiometric quantities and, more particularly, in a slight excess of the dimethyl carbonate or diethyl carbonate.

In one embodiment of the two-step process, compound (II) is introduced first in step 1 and dimethyl carbonate or diethyl carbonate is added in portions or continuously while the compound CH₃—O—CO—Z formed where dimethyl carbonate is used or the compound CH₃—CH₂—O—CO—Z formed where diethyl carbonate is used is continuously distilled off. In another embodiment of the two-step process, compound (IV) is added in portions or continuously in step 2 while the compound X—O—CO—Z formed is continuously distilled off. These last two embodiments are advantageously combined.

In another embodiment of the two-step process, reactant (IV) may optionally be used in excess in step 2 in order to obtain a higher degree of conversion. On completion of the reaction, this excess may be distilled off in vacuo.

Examples of particularly suitable groups Z in the compounds (II) are hydrogen, methyl, ethyl, n-propyl and i-propyl. Methyl is most particularly preferred, in which case (II) is triacetin (=triacetic acid ester of glycerol).

Examples of suitable groups X in the compounds (IV) are methyl, ethyl, n-propyl, i-propyl, n-butyl, i-butyl, tert.butyl, n-pentyl, n-hexyl, n-heptyl, n-octyl, 2- ethylhexyl. In a preferred embodiment, X in formula (IV) is a methyl group.

Examples of suitable groups R in the compounds (IV) are hydrogen, methyl, ethyl, n-propyl, i-propyl, n-butyl, i-butyl, tert.butyl, n-pentyl, n-hexyl, n-heptyl, n-octyl, 2-ethylhexyl, n-nonyl, n-decyl, n-undecyl, n-dodecyl, n-tridecyl, n-tetradecyl, n-pentadecyl, n-hexadecyl, n-heptadecyl, n-heptadecenylt, n-octadecyl, n-nonadecyl, n-eicosyl, n-heneicosyl, n-docosyl and —C(CH₃)═CH₂ or —CH═CH₂. R in the compounds (IV) is preferably derived from fatty acids containing 3 to 24 carbon atoms.

In another preferred embodiment, X in formula (II) is a methyl group and R is an n-heptyl group. In another preferred embodiment, X in formula (IV) is a methyl group and R is a group —C(CH₃)═CH₂ or —CH═CH₂.

The compounds (IV) may be used individually or in admixture with one another.

If desired, the process according to the invention may be carried out in the presence of a solvent. However, it is preferably carried out in the absence of a solvent.

If desired, the target product (I) may be subjected to purification steps familiar to the expert in order to increase the purity of the product.

The present invention also relates to the use of the compounds (I) as solvents or as high-boiling working liquids. It is of advantage in this regard that the compounds (i) can give off CO₂ through decomposition at elevated temperature (flameproofing effect). In addition, the compounds (I) may be used for the following purposes: hydrophobic emulsifiers, halogen-free lubricant additives, for example for fuels.

### EXAMPLES

### Example 1

### Production of Glycerol Carbonate Methacrylate

The reaction was carried out by the one-pot method, a compound (II) (in this case triacetin), dimethyl carbonate and a compound (III) (in this case methyl methacrylate) being reacted with one another at one and the same time.

The following quantities were used:

| | Mol. weight | Quantity | Quantity |
|---|---|---|---|
| | (g/mol) | (mol) | (g) |
| Triacetin | 218 | 0.5 | 109 |
| Dimethyl carbonate (DMC) | 90 | 0.5 | 45 |
| NaOMe | 54 | 0.02 | 1.08 (3.60 g |
| | | | of a 30% |
| | | | solution) |
| Methyl methacrylate (MMA) | 100 | 0.5 | 50 |
| HCl (2N) | 36.5 | 0.02 | 10.2 |

### Procedure:

Triacetin, DMC, MMA and NaOMe were stirred and heated to distil off AcOMe. The distillation was carried out at a bottom temperature of 75 to 110° C. In order to improve the yield, the distillation was continued in vacuo (from 500 to 50 mbar and up to a bottom temperature of 100° C.). The mixture was neutralized with HCl and phase separation was carried out in the presence of heat. The organic phase was washed out with 28 g water and dried.

81.2 g of product were obtained (=87% yield). Purity (GC): 67% glycerol carbonate methacrylate. The product also contained ca. 27% glycerol carbonate acetate.

### Example 2

### Production of Glycerol Carbonate Octanoate

The reaction was carried out by the one-pot method in two steps. First glycerol carbonate acetate (step 1) and then the C₈ derivative (step 2) were produced. The reaction may be schematized as follows:

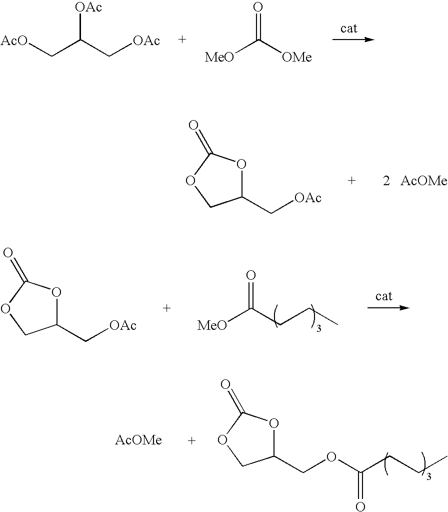

The following quantities were used:

| | Mol. weight | Quantity | Quantity | |
|---|---|---|---|---|
| | (g/mol) | (mol) | (g) | |
| Triacetin | 218 | 1 | 218 | |
| Dimethyl carbonate | 90 | 1.1 | 99 | 10% |
| | | | | excess |
| NaOMe | 54 | 0.059 | 3.19 (10.6 g | |
| | | | of a 30% | |
| | | | solution) | |
| C₈ methyl ester | 158.2 | 1.1 | 175 | 10% |
| | | | | excess |
| HCl (2N) | 36.5 | 0.059 | 30 | |
| NaHCO₃ | 84 | 0.059 | 5 (50 g | |
| | | | of a 10% | |
| | | | solution) | |
| Saturated NaCl | — | — | 50.5 + 50.5 | |
| solution | | | | |

### Procedure:

First stage. DMC, triacetin and NaOMe were heated under nitrogen to distil off AcOMe. The distillation was carried out at a bottom temperature of 62 to 100° C. Quantity of distillate: 159.5 g (theoretical quantity: 164.4 g).
Second stage. C₈ methyl ester (methyl ester of n-octanoic acid) was added. AcOMe was distilled off in a water jet vacuum at a bottom temperature of 40 to 100° C.

The mixture was neutralized with HCl. The acidic mixture was re-neutralized with a 10% NaHCO₃ solution. A first phase separation was carried out in the presence of heat. 50.5 g of a saturated NaCl solution were added and, after shaking, another phase separation was carried out. Total quantity of aqueous phase removed: 142.6 g (total quantity of water added: 130.5 g). The organic phase was washed out once more with 50.5 g saturated NaCl solution and the product was dried in a water jet vacuum at a bottom temperature of 40 to 80° C.

164 g (=67% yield) glycerol carbonate octanoate with a gas-chromatographically determined purity of 90% were obtained.

We claim:

1. A process for the production of glycerol carbonate esters of formula: 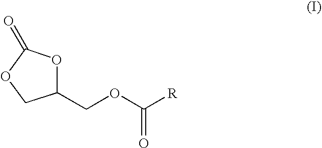
in which R is hydrogen or a linear branched alkyl or alkenyl group containing 1 to 23 carbon atoms, which comprises: transesterifying a compound of formula: 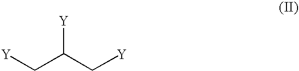
in which Y is a —O—CO—Z group and Z is hydrogen or a linear or branched alkyl or alkenyl group containing 1 to 3 carbon atoms, with at least one member selected from the group consisting of dimethyl carbonate and diethyl carbonate and an alkyl ester of formula: 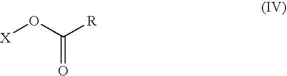
in which R is as defined above and X is a linear or branched alkyl or alkenyl group containing 1 to 8 carbon atoms, with the proviso that the group R of compound (IV) is different from the group Z of compound (II), in the presence of a transesterification catalyst.

2. A process for the production of glycerol carbonate esters of formula: 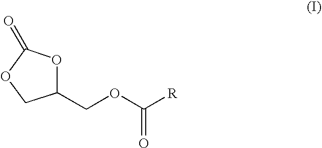
in which R is hydrogen or a linear or branched alkyl or alkenyl group containing 1 to 23 carbon atoms, which comprises:
(a) transesterifying a compound of formula: 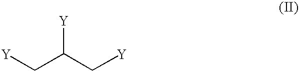
in which Y is a —O—CO—Z group and Z is hydrogen or a linear or branched alkyl or alkenyl group containing 1 to 3 carbon atoms, with at least one member selected from the group consisting of dimethyl carbonate and diethyl carbonate to form a compound of formula: 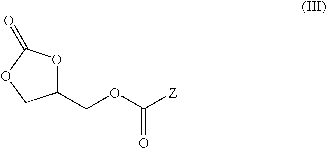
in which Z is as defined above; and,
(b) transesterifying compound (III) with an alkyl ester of formula: 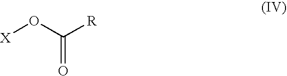
in which R is as defined above and X is a linear or branched alkyl or alkenyl group containing 1 to 8 carbon atoms, with the proviso that the group R of compound (IV) is different from the group Z of compound (II), in the presence of a transesterification catalyst.

3. At least one member selected from the group consisting of solvents, working liquids with a flameproofing effect and lubricant additives for fuels comprising a compound of formula I.

4. The process of claim 1, wherein, the transesterification is carried out at a temperature of from 40° C. to 150° C.

5. The process of claim 2, wherein, the transesterification is carried out at a temperature of from 40° C. to 150° C.

6. The process of claim 1, wherein, R is a —C(CH₃)═CH₂ or —CH₂═CH₂.

7. The process of claim 2, wherein, R is a —C(CH₃)═CH₂ or —CH₂═CH₂.

8. The process of claim 1, wherein, the transesterification catalyst comprises at least one member selected from the group consisting of sodium methylate, potassium methylate, alkali metal hydroxides and alkali metal carbonates.

9. The process of claim 2, wherein, the transesterification catalyst comprises at least one member selected from the group consisting of sodium methylate, potassium methylate, alkali metal hydroxides and alkali metal carbonates.
